(19) United States
(12) Patent Application Publication
(10) Pub. No.: US 2025/0099692 A1
(43) Pub. Date: Mar. 27, 2025

(54) METERED-DOSE ATOMIZATION MODULE, ATOMIZER, SPRAY ASSEMBLY AND USE THEREOF

(71) Applicant: SUNSHINE LAKE PHARMA CO., LTD., Dongguan, Guangdong (CN)

(72) Inventors: Xuqi LIN, Dongguan (CN); Sumin HE, Dongguan (CN); Liqiu MOU, Dongguan (CN); Li CAO, Dongguan (CN); Jinsong YOU, Dongguan (CN); Fangfang HUANG, Dongguan (CN)

(73) Assignee: SUNSHINE LAKE PHARMA CO., LTD., Dongguan, Guangdong (CN)

(21) Appl. No.: 18/724,815

(22) PCT Filed: Dec. 27, 2022
(86) PCT No.: PCT/CN2022/142224

§ 371 Date: Jun. 27, 2024

(30) Foreign Application Priority Data
Dec. 28, 2021 (CN) 202111629761.X

(51) Int. Cl. A61M 11/00

(52) U.S. Cl. CPC A61M 11/001

(57) ABSTRACT

A metered-dose atomization module, an atomizer, a spray assembly and the use thereof. The metered-dose atomization module includes: a spray head in which an atomization cavity has a step surface; an atomization piece, including an annular brake and a microporous membrane attached to a side surface of the annular brake; rubber rings, including a first sealing ring and a second sealing ring, the first sealing ring and the second sealing ring being annular and arranged on two sides of the atomization piece respectively; and a pressing plate, the pressing plate being annular and fixed to the spray head, and being adapted to cooperate with the step surface to install the atomization piece and the rubber rings in the atomization cavity.

### CROSS-REFERENCE TO RELATED APPLICATION

This is a U.S. national stage application of the International Patent Application No. PCT/CN2022/142224, filed on Dec. 27, 2022, which claims the priority and benefits of Chinese Patent Application No.s 202111629761.X, filed with the State Intellectual Property Office of China on Dec. 28, 2021, which is incorporated herein by reference in their entirety.

### FIELD OF THE INVENTION

The invention belongs to the technical field of medical devices, and specifically relates to a metered-dose atomization module, an atomizer, a spray assembly and uses thereof.

### BACKGROUND

Vibrating mesh atomizers usually atomize in a manner similar to a predetermined amount, all the drug in the packaging material are poured into the atomization cavity of the atomizer when using, and then the mesh atomization piece is vibrated for several to tens of minutes to atomize the drug to achieve drug delivery. In traditional vibrating mesh atomizers, the atomization piece is usually wrapped by a half-wrapped silicone gasket and placed in the atomization cavity, and then pressure is applied to the silicone gasket through the top shell to seal the atomization piece to the atomization cavity. In the above structure, due to the poor dimensional accuracy and consistency of the silicone gasket, the atomization piece will move radially relative to the silicone gasket due to unbalanced forces during vibration when used for a long time, thus affecting the sealing effect. Therefore, it is usually necessary to increase the pressure exerted by the top shell to suppress the radial movement of the atomization piece; however, a larger external force will also reduce the axial vibration of the atomization piece, thereby reducing the atomization rate.

### DETAILED DESCRIPTION OF THE INVENTION

The object of the present invention is to provide a metered-dose atomization module, a metered-dose atomizer, a spray assembly and uses thereof to solve the above problems existing in the prior art.

The metered-dose atomization module provided herein comprises: a spray head, which is provided with an atomization cavity with a step surface; an atomization piece, which is located in the atomization cavity and comprises an annular brake and a microporous membrane, the microporous membrane is attached to the side surface of the annular brake; rubber rings, comprising a first sealing ring and a second sealing ring, the first sealing ring and the second sealing ring are both annular and clamped on both sides of the atomization piece respectively; and a pressing plate, the pressing plate is annular and fixed with the spray head, and is suitable for fixing the atomization piece and the rubber rings on the step surface of the atomization cavity.

The metered-dose atomization module as described above is further preferred that the microporous membrane comprises a central area covering the central hole of the annular brake, and an adhesive area connected with the annular brake; and the internal stress of the microporous membrane remains constant before and after connecting.

The metered-dose atomization module as described above is further preferred that the central area comprises a micropore area densely covered with micropores, and an outer ring area surrounding the periphery of the micropore area, the total surface curvature change of the micropore area is larger than that of the outer ring area.

The metered-dose atomization module as described above is further preferred that the microporous membrane is made of stainless steel material, and the Vickers hardness of the stainless steel material is 200-500 HV.

The metered-dose atomization module as described above is further preferred that the annular brake is piezoelectric ceramic, the piezoelectric constant of the piezoelectric ceramic is 200-800 pC/N, and the mechanical quality factor is 50-1200.

The metered-dose atomization module as described above is further preferred that the upper and lower surfaces of the first sealing ring are both provided with annular protrusions, and the protrusions are suitable for sealing with the step surface of the atomization cavity or the microporous membrane.

The metered-dose atomization module as described above is further preferred that the side surface of the pressing plate is further provided with a positioning ring coaxially arranged with the pressing plate; the outer diameter of the positioning ring is larger than the outer diameter of the second sealing ring, and the inner diameter is smaller than the inner diameter of the second sealing ring, which is suitable for corresponding press fit with the first sealing ring.

The present invention also provides an atomizer, comprising the metered-dose atomization module described in any one of the above.

The atomizer as described above is further preferred that, further comprising: a medicine bottle, which is detachably connected with the inlet of the atomization cavity in the spray head, a shell, comprising a curved neck shell and a main shell, the curved neck shell is located on the outside of the medicine bottle, and one end is connected with the spray head, and the other end is connected with the main shell; and a driving device, which is located in the main shell and is electrically connected with the atomization piece.

The atomizer as described above is further preferred that the driving frequency of the driving device is the natural frequency of the atomization piece, and the output voltage RMS is in the range of 15-30V.

The present invention also provides a spray assembly, comprising spray and the aforementioned atomizer, the active pharmaceutical ingredient in the spray comprises at least one of β2 receptor agonist, glucocorticoid, muscarinic receptor antagonist and phosphodiesterase 4 inhibitor. In some embodiments, the spray assembly comprises a single spray of β2 receptor agonist. In some embodiments, the spray assembly comprises a single spray of glucocorticoid. In some embodiments, the spray assembly comprises a single spray of muscarinic receptor antagonist. In some embodiments, the spray assembly comprises a single spray of phosphodiesterase 4 inhibitor. In some embodiments, the spray assembly comprises a combination spray of β2 receptor agonist and glucocorticoid. In some embodiments, the spray assembly comprises a combination spray of β2 receptor agonist and muscarinic receptor antagonist. In some embodiments, the spray assembly comprises a combination spray of muscarinic receptor antagonist and glucocorticoid. In some embodiments, the spray assembly comprises a three-part spray of β2 receptor agonist, muscarinic receptor antagonist and glucocorticoid.

In some embodiments, the β2 receptor agonist comprises at least one of salbutamol or a pharmaceutically acceptable salt thereof, fenoterol or a pharmaceutically acceptable salt thereof, terbutaline or a pharmaceutically acceptable salt thereof, formoterol or a pharmaceutically acceptable salt thereof, olodaterol or a pharmaceutically acceptable salt thereof, arformoterol or a pharmaceutically acceptable salt thereof, indacaterol or a pharmaceutically acceptable salt thereof, vilanterol or a pharmaceutically acceptable salt thereof. The pharmaceutically acceptable salt of salbutamol may be salbutamol sulfate or salbutamol hydrochloride. The pharmaceutically acceptable salt of formoterol may be formoterol fumarate. The pharmaceutically acceptable salt of olodaterol may be olodaterol hydrochloride. The pharmaceutically acceptable salt of arformoterol may be arformoterol tartrate. The pharmaceutically acceptable salt of indacaterol may be indacaterol maleate. The pharmaceutically acceptable salt of vilanterol may be vilanterol triphenylacetate.

In some embodiments, the glucocorticoid comprises at least one of fluticasone or a pharmaceutically acceptable salt or ester thereof, mometasone or a pharmaceutically acceptable salt or ester thereof, ciclesonide or a pharmaceutically acceptable salt or ester thereof, beclomethasone or a pharmaceutically acceptable salt or ester thereof, flunisolide or a pharmaceutically acceptable salt or ester thereof, budesonide or a pharmaceutically acceptable salt or ester thereof, triamcinolone acetonide or a pharmaceutically acceptable salt or ester thereof, dexamethasone or a pharmaceutically acceptable salt or ester thereof. The pharmaceutically acceptable salt of fluticasone may be fluticasone furoate or fluticasone propionate. The pharmaceutically acceptable salt of mometasone may be mometasone furoate. The pharmaceutically acceptable salt of beclomethasone may be beclomethasone dipropionate or beclomethasone propionate. The pharmaceutically acceptable salt of dexamethasone may be dexamethasone sodium phosphate.

In some embodiments, the muscarinic receptor antagonist comprises at least one of tiotropium or a pharmaceutically acceptable salt thereof, glycopyrronium or a pharmaceutically acceptable salt thereof, umeclidinium or a pharmaceutically acceptable salt thereof, aclidinium or a pharmaceutically acceptable salt thereof, ipratropium or a pharmaceutically acceptable salt thereof, oxitropium or a pharmaceutically acceptable salt thereof, revefenacin or a pharmaceutically acceptable salt thereof. The pharmaceutically acceptable salt of tiotropium may be tiotropium bromide. The pharmaceutically acceptable salt of glycopyrronium may be glycopyrrolate. The pharmaceutically acceptable salt of umeclidinium may be umeclidinium bromide. The pharmaceutically acceptable salt of aclidinium may be aclidinium bromide. The pharmaceutically acceptable salt of ipratropium may be ipratropium bromide. The pharmaceutically acceptable salt of oxitropium may be oxitropium bromide.

In some embodiments, the phosphodiesterase 4 inhibitor comprises at least one of roflumilast or a pharmaceutically acceptable derivative thereof, apremilast or a pharmaceutically acceptable derivative thereof. The pharmaceutically acceptable derivative of roflumilast may be roflumilast N-oxide.

The present invention also provides a spray assembly comprising spray and the aforementioned atomizer, the active pharmaceutical ingredient in the spray comprises at least one of prostacyclin, treprostinil and iloprost.

The present invention also provides a spray assembly comprising spray and the aforementioned atomizer, and the active pharmaceutical ingredient in the spray comprises antibiotic or an antiviral drug. The antibiotic comprises at least one of aztreonam, tobramycin, amikacin, and ciprofloxacin, and the antiviral drug comprises at least one of zanamivir, Laninamivir, and ribavirin.

The present invention also provides a spray assembly comprising spray and the aforementioned atomizer, and the active pharmaceutical ingredient in the spray comprises at least one of pirfenidone and nintedanib.

The present invention also provides a spray assembly comprising spray and the aforementioned atomizer, and the active pharmaceutical ingredient in the spray comprises a small molecule cytotoxic drug or a biological agent. The small molecule cytotoxic drug comprises at least one of cisplatin, cyclophosphamide, etoposide, vinorelbine, and paclitaxel, and the biological agent comprises at least one of ipilimumab, nivolumab, and durvalumab.

According to one aspect of the present invention, the invention provides a method for treating COPD (chronic obstructive pulmonary disease) and/or asthma in a human in need thereof, wherein the method comprises administering to the human a spray assembly.

In some embodiments, the invention provides a method for treating pulmonary arterial hypertension in a human in need thereof, wherein the method comprises administering to the human a spray assembly.

According to one aspect of the present invention, the invention provides a method for treating lung infection in a human in need thereof, wherein the method comprises administering to the human a spray assembly.

According to one aspect of the present invention, the invention provides a method for treating idiopathic pulmonary fibrosis in a human in need thereof, wherein the method comprises administering to the human a spray assembly.

In some embodiments, According to one aspect of the present invention, the invention provides a method for treating idiopathic pulmonary fibrosis in a human in need thereof, wherein the method comprises administering to the human a spray assembly.

According to another aspect of the present invention, the aforementioned spray assembly can be used for systemic administration of small molecule drugs such as levodopa and loxapine through the lungs; it can also be used for the systemic administration of biological agents such as insulin and insulin analogs through the lungs.

The invention provides a metered-dose atomization module, and specifically comprises a spray head, an atomization piece, rubber rings and a pressing plate, wherein the spray head is provided with an atomization cavity with a step surface, and the atomization piece is located in the atomization cavity, comprising an annular brake and a microporous membrane, the microporous membrane is attached to the side surface of the annular brake; the rubber rings comprise a first sealing ring and a second sealing ring, both of which are annular, and are clamped on both sides of the atomization piece respectively; the pressing plate is annular and fixed with the spray head, and being adapted to cooperate with the step surface to fix the atomization piece and the rubber rings in the atomization cavity. That is, the corresponding installation of the atomization piece and the atomization cavity is realized through the above structure, and the installation of the atomization piece is stable, thereby maintaining good vibration consistency, so that the metered-dose atomization module has a stable and high atomization rate.

Based on the above structure, the metered-dose mesh atomizer can quantitatively atomize 5 μL-60 μL of spray in 1 s-3 s, the percentage of droplets with an aerodynamic particle size of less than 5.8 μm in the spray to the total mass of the droplets is more than 65%. The above-mentioned quantitative standard is that the single value and average value of the atomization volume of the mesh atomizer for multiple days do not exceed 10% of the average value of all days.

### DESCRIPTION OF THE DRAWINGS

In order to more clearly explain the specific embodiments of the present invention or the technical solutions in the prior art, the figures that need to be used in the description of the specific embodiments or the prior art will be briefly introduced below. Obviously, the figures in the following description are some embodiments of the present invention, for those of ordinary skill in the art, other figures can be obtained based on these figures without exerting creative efforts.

FIG. 1 is a schematic cross-sectional view of the metered-dose atomization module in the present invention;

FIG. 2 is a schematic structural diagram of the atomization piece in the present invention;

FIG. 3 is a schematic structural diagram of the microporous membrane;

FIG. 4 is a schematic structural diagram of the pressing plate;

FIG. 5 is a schematic structural diagram of the first sealing ring;

FIG. 6 is a schematic structural diagram of the atomizer in the present invention;

FIG. 7 is a partial enlarged view of the atomizer in the figure;

FIG. 8 is a displacement change diagram of the nodes in the atomization piece;

FIG. 9 is the schematic diagrams of the center displacement and time domain results of the micropore areas in the atomization piece, and the spectrum diagrams after Fourier transform. Wherein FIG. 9a, FIG. 9b, and FIG. 9c are schematic diagrams of the center displacement and the time domain results of the micropore areas of the three atomization pieces, respectively; FIG. 9d, FIG. 9e, and FIG. 9f are spectrum diagrams after Fourier transform of the micropore areas of the three atomization pieces, respectively.

### EXPLANATION OF FIGURE SYMBOLS

1—Spray head, 2—Atomization cavity, 3—Atomization piece, 4—First sealing ring, 5—Second sealing ring, 6—Positioning ring, 7—Pressing plate, 8—Medicine bottle, 9—Shell;
31—Annular brake, 32—Microporous membrane; 321—Microporous area, 322—Outer ring area, 323—Adhesive area, 324—Fixing arm;
41—Protrusion;
91—Curved neck shell, 92—Main shell, 93—Bottle cap, 94—Bottle stopper.

### EXAMPLES

The technical solution of the present invention will be clearly and completely described below with reference to the accompanying figures. Obviously, the described embodiments are some, not all, of the embodiments of the present invention. Based on the embodiments of the present invention, all other embodiments obtained by those of ordinary skill in the art without creative efforts fall within the scope of protection of the present invention.

In the description of the present invention, it should be noted that the terms “center”, “upper”, “lower”, “left”, “right”, “vertical”, “horizontal”, “inner”, “outer”, etc. indicating orientation or positional relationship is based on the orientation or positional relationship shown in the figures, it is only for the convenience of describing the present invention and simplifying the description, rather than indicating or implying that the device or component referred to must have a specific orientation, be constructed and operate in a specific orientation, and therefore it should not be construed as limitations of the invention. Furthermore, the terms “first”, “second” and “third” are used for descriptive purposes only and are not to be construed as indicating or implying relative importance.

In the description of the present invention, it should be noted that, unless otherwise clearly stated and limited, the terms “install”, “installation”, “connection” and “connect” should be understood in a broad sense. For example, it can be a fixed connection or a detachable connection, or an integral connection; it can be a mechanical connection or an electrical connection; it can be a direct connection or an indirect connection through an intermediate medium; it can be an internal connection between two components. For those of ordinary skill in the art, the specific meanings of the terms in the present invention can be understood on a case-by-case basis.

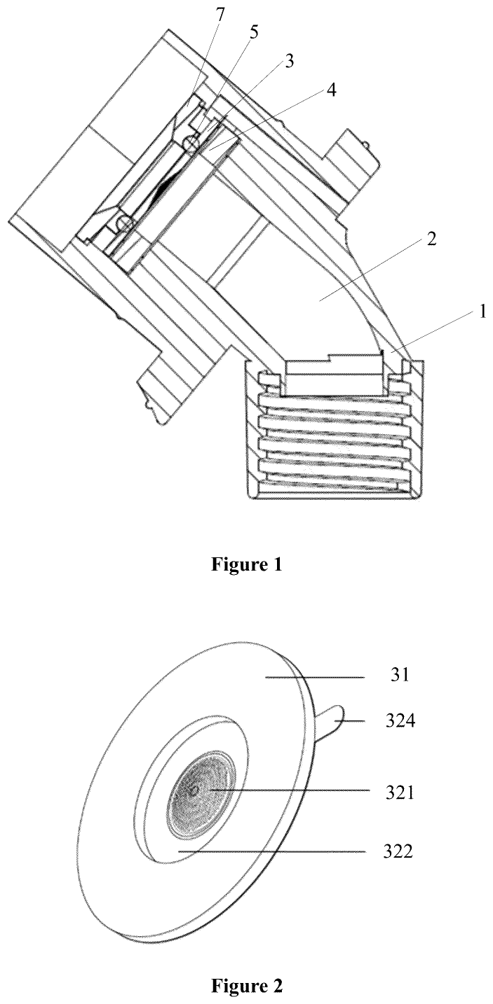

The metered-dose atomization module and the atomizer in some embodiments of the present invention will be described below with reference to FIGS. 1-7.

Referring to FIGS. 1-5, the metered-dose atomization module disclosed in this embodiment mainly comprises a spray head 1, an atomization piece 3, rubber rings and a pressing plate 7, wherein the spray head 1 is provided with an atomization cavity 2 with a step surface, the atomization piece 3 is arranged in the atomization cavity 2 and comprises an annular brake 31 and a microporous membrane 32, the microporous membrane 32 is attached to the side surface of the annular brake 31; the rubber rings comprises a first sealing ring 4 and a second sealing ring 5, the first sealing ring 4 and the second sealing ring 5 are both annular and clamped on both sides of the atomization piece 3 respectively; the pressing plate 7 is annular and fixed with the spray head 1, and being adapted to cooperate with the step surface to fix the atomization piece 3 and the rubber rings in the atomization cavity 2. That is, the corresponding installation of the atomizing sheet 3 and the atomizing chamber 2 is achieved through the above structure.

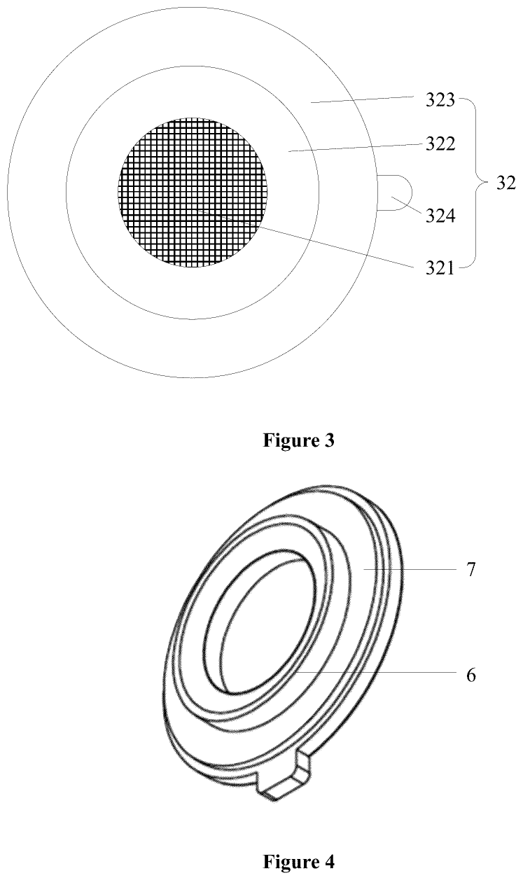

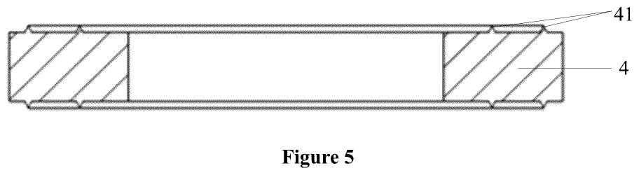

Referring to FIGS. 2-3, in the atomization piece 3, the microporous membrane 32 comprises a central area covering the central hole of the annular brake 31, and an adhesive area 323 connected with the annular brake 31; and the internal stress of the microporous membrane remains constant before and after connecting. Specifically, during the connecting operation, the microporous membrane is kept flat and does not deform or only deforms invisible to the naked eye, so that the stress in the central area does not change. The central area comprises a micropore area 321 densely covered with micropores, and an outer ring area 322 surrounding the periphery of the micropore area 321, the surface curvature change of the micropore area 321 is larger than that of the outer ring area 322. Specifically, by processing the shape of the microporous membrane, there is a node at the boundary between the microporous area 321 of the central area and the outer ring area 322 at a specific frequency, this node reduces the curvature of the surface, creating an area that can be heavily fogged.

In addition, a fixing arm 324 is provided outside the adhesive area. Preferably, the microporous membrane 32 is made of stainless steel material, and the Victoria hardness of the stainless steel material is 200-500 HV.

In the atomization piece 3, the annular brake 31 is piezoelectric ceramic, the piezoelectric constant of the piezoelectric ceramic is 200-800 pC/N, and the mechanical quality factor is 50-1200. The brake vibrates under the drive of a specific frequency, thereby driving the microporous membrane 32 to vibrate together, when the microporous membrane 32 vibrates, the surface of the central area deforms, causing the volume of the micropores in the microporous area 321 to change to produce a pump effect, and the droplets are ejected to form fog.

By setting the formation parameters of the microporous membrane material in the atomization piece and the performance parameters of the brake material, the microporous area 321 of the atomization piece has a specific and stable displacement, and then contacts and connects with the medicine liquid to vibrate and atomize a quantitative amount of the medicine liquid.

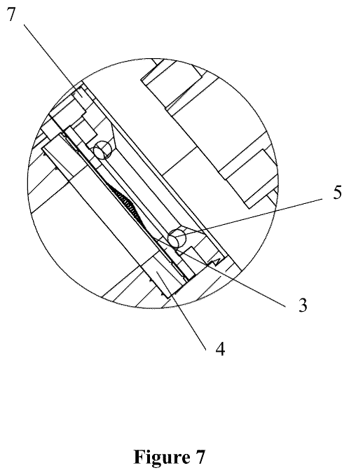

Referring to FIG. 5, among the rubber rings, annular protrusions 41 are provided on both the upper and lower surfaces of the first sealing ring 4, the protrusions 41 are suitable for sealing with the step surface of the atomization cavity 2 or the annular brake 31. Specifically, the first sealing ring 4 is provided on the lower surface of the atomization piece 3 and is suitable for sealing the atomization piece 3 and the step surface of the atomization cavity 2. The protrusions 41 are an annular protrusion, and the upper surface and the lower surface are respectively provided with two, and are respectively located on the annular surface between ½D-¾D (D is the diameter) and the annular surface between ¾D-D. In addition to increasing the sealing performance, the protrusions 41 can also reduce the dynamic friction of the second sealing ring 5 on the brake, and reduce the displacement of the atomization piece 3 when it vibrates.

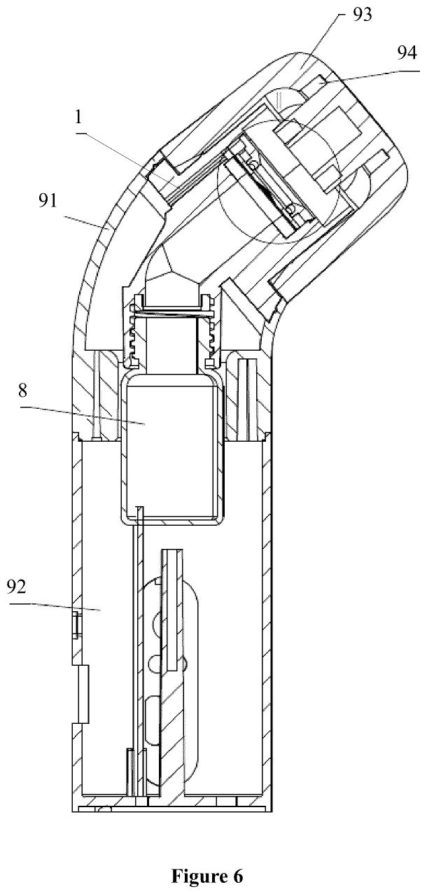

Referring to FIG. 4, the side surface of the pressing plate is also provided with a positioning ring 6 arranged coaxially with the pressing plate; the outer diameter of the positioning ring 6 is not less than the outer diameter of the second sealing ring, and the inner diameter is not greater than the inner diameter of the second sealing ring, and is suitable for corresponding press fit with the first sealing ring. The outer diameter of the pressing plate is smaller than the diameter of the atomization cavity and is arranged in the atomization cavity, the positioning ring is arranged on the side surface of the pressing plate, and the outer diameter is smaller than the outer diameter of the pressing plate, so that an avoidance space is formed around the outer periphery of the positioning ring to facilitate the corresponding connection between the pressing plate and the spray head. The inner diameter of the positioning ring 6 is less than or equal to the inner diameter of the second sealing ring 5 and larger than the inner diameter of the micropore area 321, so that when the annular surface of the positioning ring cooperates with the second sealing ring, the positioning of the second sealing ring 5 in both axial and radial directions can achieve. In the atomization cavity, the outer diameter of the step surface is greater than or equal to the outer diameter of the first sealing ring, and the inner diameter is less than or equal to the inner diameter of the first sealing ring and greater than the inner diameter of the micropore area 321, which is suitable for forming a complete constraint with the lower surface of the first sealing ring. Through the above size limitation, when the pressing plate and the step surface are matched to install the atomization piece and the rubber ring in the atomization cavity, the static friction and dynamic friction of the annular brake in the radial direction can be reduced, and the strain suppression effect on the annular brake can be reduced.

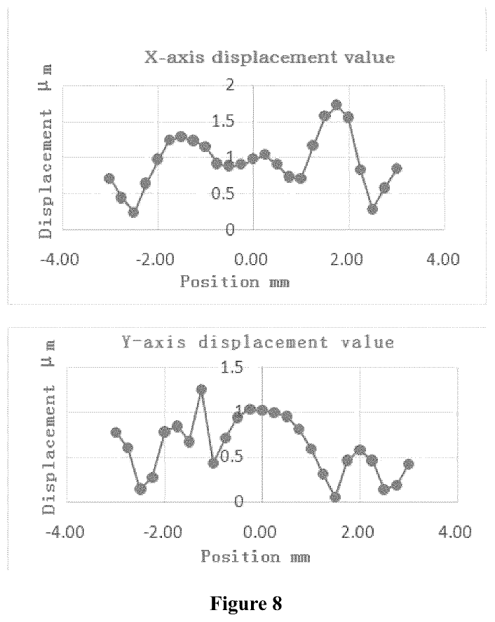

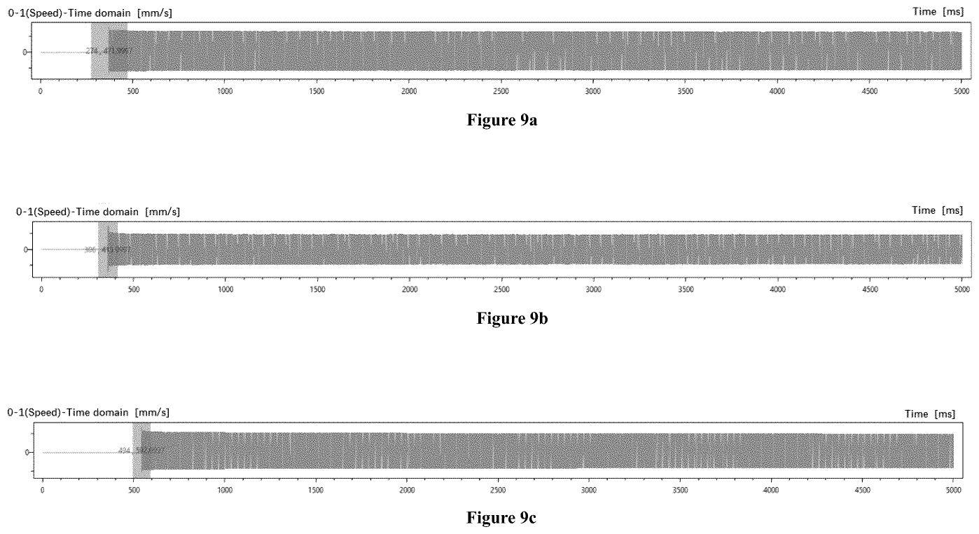

Referring to FIGS. 6-7, this embodiment also provides an atomizer, which comprises the metered-dose atomization module as described above, further more, it also comprises a medicine bottle 8, a shell 9 and a driving device, wherein the medicine bottle 8 is detachably connected with the inlet of the atomization cavity 2 in the spray head 1; the shell 9 comprises a curved neck shell 91 and a main shell 92, the curved neck shell 91 is located on the outside of the medicine bottle 8, and one end is connected with the spray head 1, and the other end is connected with the main shell 92; the driving device is located in the main shell 92 and is electrically connected with the atomization piece 3. Preferably, the driving device drives the atomization piece to vibrate using the natural frequency of the atomization piece as the driving frequency. Specifically, the curved neck shell 91 is detachably connected with the spray head 1 and the main shell 92. On the one hand, it is convenient for the replacement of the metered-dose atomization module, and on the other hand, it is convenient to disassemble and assemble the medicine bottle 8. In addition, the above-mentioned atomizer also comprises a bottle cap 93 and a bottle stopper 94 located in the bottle cap 93, the bottle cap 93 is adapted to the spray head 1, the bottle stopper 94 is adapted to the outlet of the atomization cavity 2, and the bottle stopper 94 is suitable for blocking the atomization cavity 2 when the bottle cap 93 is adapted and installed with the spray head 1.

Based on the above structure, the atomizer can quantitatively atomize 5 μL-60 μL of spray in 1 s-3 s, and the percentage of droplets with an aerodynamic particle size of less than 5.8 μm in the spray is more than 65% of the total mass of the droplets.

In some embodiments, the percentage of droplets with an aerodynamic particle size of less than 5.8 μm in the spray to the total mass of the droplets is more than 70%. In some embodiments, the percentage of droplets with an aerodynamic particle size of less than 5.8 μm in the spray to the total mass of the droplets is more than 75%. In some embodiments, the percentage of droplets with an aerodynamic particle size of less than 5.8 μm in the spray to the total mass of the droplets is more than 60%. In some embodiments, the percentage of droplets with an aerodynamic particle size of less than 5.8 μm in the spray to the total mass of the droplets is more than 65%.

The aerodynamic particle size refers to a certain type of particle, regardless of its shape, size and density, if its settling speed in the air is consistent with the settling speed of a spherical particle with a density of 1, then the diameter of a spherical particle is the aerodynamic particle size of the particle.

The present invention also provides a spray assembly, comprising spray and the aforementioned atomizer, the active pharmaceutical ingredient in the spray are at least one of 2 receptor agonist, glucocorticoid, muscarinic receptor antagonist and phosphodiesterase 4 inhibitor. In some embodiments, the spray assembly comprises a single spray of β2 receptor agonist. In some embodiments, the spray assembly comprises a single spray of glucocorticoid. In some embodiments, the spray assembly comprises a single spray of muscarinic receptor antagonist. In some embodiments, the spray assembly comprises a single spray of phosphodiesterase 4 inhibitor. In some embodiments, the spray assembly comprises a combination spray of β2 receptor agonist and glucocorticoid. In some embodiments, the spray assembly comprises a combination spray of β2 receptor agonist and muscarinic receptor antagonist. In some embodiments, the spray assembly comprises a combination spray of muscarinic receptor antagonist and glucocorticoid. In some embodiments, the spray assembly comprises a three-part spray of β2 receptor agonist, muscarinic receptor antagonist and glucocorticoid.

In some embodiments, the β2 receptor agonist comprises at least one of salbutamol or a pharmaceutically acceptable salt thereof, fenoterol or a pharmaceutically acceptable salt thereof, terbutaline or a pharmaceutically acceptable salt thereof, formoterol or a pharmaceutically acceptable salt thereof, olodaterol or a pharmaceutically acceptable salt thereof, arformoterol or a pharmaceutically acceptable salt thereof, indacaterol or a pharmaceutically acceptable salt thereof, vilanterol or a pharmaceutically acceptable salt thereof. The pharmaceutically acceptable salt of salbutamol may be salbutamol sulfate or salbutamol hydrochloride. The pharmaceutically acceptable salt of formoterol may be formoterol fumarate. The pharmaceutically acceptable salt of olodaterol may be olodaterol hydrochloride. The pharmaceutically acceptable salt of arformoterol may be arformoterol tartrate. The pharmaceutically acceptable salt of indacaterol may be indacaterol maleate. The pharmaceutically acceptable salt of vilanterol may be vilanterol triphenylacetate.

In some embodiments, the glucocorticoid comprises at least one of fluticasone or a pharmaceutically acceptable salt or ester thereof, mometasone or a pharmaceutically acceptable salt or ester thereof, ciclesonide or a pharmaceutically acceptable salt or ester thereof, beclomethasone or a pharmaceutically acceptable salt or ester thereof, flunisolide or a pharmaceutically acceptable salt or ester thereof, budesonide or a pharmaceutically acceptable salt or ester thereof, triamcinolone acetonide or a pharmaceutically acceptable salt or ester thereof, dexamethasone or a pharmaceutically acceptable salt or ester thereof. The pharmaceutically acceptable salt of fluticasone may be fluticasone furoate or fluticasone propionate. The pharmaceutically acceptable salt of mometasone may be mometasone furoate. The pharmaceutically acceptable salt of beclomethasone may be beclomethasone dipropionate or beclomethasone propionate. The pharmaceutically acceptable salt of dexamethasone may be dexamethasone sodium phosphate.

In some embodiments, the muscarinic receptor antagonist comprises at least one of tiotropium or a pharmaceutically acceptable salt thereof, glycopyrronium or a pharmaceutically acceptable salt thereof, umeclidinium or a pharmaceutically acceptable salt thereof, aclidinium or a pharmaceutically acceptable salt thereof, ipratropium or a pharmaceutically acceptable salt thereof, oxitropium or a pharmaceutically acceptable salt thereof, revefenacin or a pharmaceutically acceptable salt thereof. The pharmaceutically acceptable salt of tiotropium may be tiotropium bromide. The pharmaceutically acceptable salt of glycopyrronium may be glycopyrrolate. The pharmaceutically acceptable salt of umeclidinium may be umeclidinium bromide. The pharmaceutically acceptable salt of aclidinium may be aclidinium bromide. The pharmaceutically acceptable salt of ipratropium may be ipratropium bromide. The pharmaceutically acceptable salt of oxitropium may be oxitropium bromide.

In some embodiments, the phosphodiesterase 4 inhibitor comprises at least one of roflumilast or a pharmaceutically acceptable derivative thereof, apremilast or a pharmaceutically acceptable derivative thereof. The pharmaceutically acceptable derivative of roflumilast may be roflumilast N-oxide.

The present invention also provides a spray assembly comprising spray and the aforementioned atomizer, the active pharmaceutical ingredient in the spray comprises at least one of prostacyclin, treprostinil and iloprost.

The present invention also provides a spray assembly comprising spray and the aforementioned atomizer, and the active pharmaceutical ingredient in the spray comprises antibiotic or an antiviral drug. The antibiotic comprises at least one of aztreonam, tobramycin, amikacin, and ciprofloxacin, and the antiviral drug comprises at least one of zanamivir, Laninamivir, and ribavirin.

The present invention also provides a spray assembly comprising spray and the aforementioned atomizer, and the active pharmaceutical ingredient in the spray comprises at least one of pirfenidone and nintedanib.

The present invention also provides a spray assembly comprising spray and the aforementioned atomizer, and the active pharmaceutical ingredient in the spray comprises a small molecule cytotoxic drug or a biological agent. The small molecule cytotoxic drug comprises at least one of cisplatin, cyclophosphamide, etoposide, vinorelbine, and paclitaxel, and the biological agent comprises at least one of ipilimumab, nivolumab, and durvalumab.

According to one aspect of the present invention, the present invention provides use of the aforementioned spray assembly in the manufacture of a medicament for treating COPD (chronic obstructive pulmonary disease) and/or asthma.

In some embodiments, the present invention provides use of the aforementioned spray assembly in the manufacture of a medicament for treating pulmonary hypertension.

In some embodiments, the present invention provides use of the aforementioned spray assembly in the manufacture of a medicament for treating pulmonary infection.

In some embodiments, the present invention provides use of the aforementioned spray assembly in the manufacture of a medicament for treating idiopathic pulmonary fibrosis.

In some embodiments, the present invention provides use of the aforementioned spray assembly in the manufacture of a medicament for treating lung cancer.

According to another aspect of the present invention, the aforementioned spray assembly can be used for systemic administration of small molecule drugs such as levodopa and loxapine through the lungs; it can also be used for the systemic administration of biological agents such as insulin and insulin analogs through the lungs.

Further more, based on the above atomizer, the following tests were also conducted.

Test 1: The atomization rate test was conducted based on the performance of piezoelectric ceramics and stainless steel in the atomization piece:

### 1. Instruments and Materials

Instruments: electronic balance (specification: XPR404S, manufacturer: Mettler, Switzerland), signal generator (specification: AFG10022, manufacturer: Tektronix), power amplifier (specification: ATA-2031, manufacturer: Antai);
Materials: piezoelectric ceramics, adhesive, microporous membrane;

### 2. Experimental Steps:

The microporous membrane 32 and the annular piezoelectric ceramic were closely pasted together through the adhesive, after a period of solidification, an atomization piece 3 was formed; the water outlet hole of the atomization piece 3 was facing downward, and about 2 mL of tiotropium bromide solution was added dropwise through the water inlet surface, the atomization piece was connected with the signal source and power amplifier with adjusted parameters, the output voltage RMS was 25V, the output frequency of the atomization pieces 1-01, 1-02, 1-03, 1-04 and 2-01, 2-02, 2-03, 2-04 and 5-01, 5-02, 5-03 was 101 KHz, and the output frequency of the atomization pieces 3-01, 3-02, 3-03, 3-04, 4-01, 4-02, 4-03 was 91 KHz, and atomized regularly for 1.5 s. Weight loss method was used to measure the amount of atomization and the rate was calculated;

2. The experimental results are shown in Table 1.

TABLE 1

| | | Mechanical | Hardness of | Atomized | Atomized | Atomized | Average |
|---|---|---|---|---|---|---|---|
| Atomization | Piezoelectric | quality | microporous | amount 1 | amount 2 | amount 3 | atomization |
| piece | constant | factor | membrane | (mg) | (mg) | (mg) | rate mg/s |
| Atomization | 387 | 904.4 | 247 | 12.2 | 12.3 | 12.7 | 8.3 |
| piece 1-01 | | | | | | | |
| Atomization | 384 | 734.3 | 247 | 11.9 | 12.1 | 12.4 | 8.1 |
| piece 1-02 | | | | | | | |
| Atomization | 376 | 845.5 | 247 | 13.6 | 13.4 | 13.3 | 9.0 |
| piece 1-03 | | | | | | | |
| Atomization | 380 | 761.3 | 247 | 12.4 | 12.2 | 12.4 | 8.2 |
| piece 1-04 | | | | | | | |
| Atomization | 466 | 75.0 | 247 | 13.7 | 13.6 | 13.1 | 9.0 |
| piece 2-01 | | | | | | | |
| Atomization | 473 | 74.4 | 247 | 12.9 | 13.6 | 12.6 | 8.7 |
| piece 2-02 | | | | | | | |
| Atomization | 493 | 74.4 | 247 | 13.6 | 13.8 | 14 | 9.2 |
| piece 2-03 | | | | | | | |
| Atomization | 471 | 75.8 | 247 | 13.1 | 13.2 | 13.5 | 8.8 |
| piece 2-04 | | | | | | | |
| Atomization | 607 | 77.9 | 247 | 17.6 | 17.6 | 17.5 | 11.7 |
| piece 3-01 | | | | | | | |
| Atomization | 572 | 76.4 | 247 | 17.2 | 17 | 17.2 | 11.4 |
| piece 3-02 | | | | | | | |
| Atomization | 641 | 76.2 | 247 | 17.1 | 17.4 | 17.5 | 11.6 |
| piece 3-03 | | | | | | | |
| Atomization | 637 | 72.7 | 247 | 17.7 | 17.9 | 17.5 | 11.8 |
| piece 3-04 | | | | | | | |
| Atomization | 458 | 72.1 | 352 | 8.6 | 8.7 | 8.8 | 5.8 |
| piece 4-01 | | | | | | | |
| Atomization | 471 | 77.3 | 352 | 8 | 8 | 7.8 | 5.3 |
| piece 4-02 | | | | | | | |
| Atomization | 456 | 74.4 | 352 | 8.5 | 8.8 | 8.8 | 5.8 |
| piece 4-03 | | | | | | | |
| Atomization | 382 | 766.1 | 352 | 16.9 | 17.6 | 17.1 | 11.5 |
| piece 5-01 | | | | | | | |
| Atomization | 377 | 784.0 | 352 | 16.7 | 16.6 | 16.6 | 11.1 |
| piece 5-02 | | | | | | | |
| Atomization | 391 | 761.7 | 352 | 16.7 | 16.9 | 17 | 11.3 |
| piece 5-03 | | | | | | | |

When using materials (ceramics and stainless steel) within the range of material properties described above, the atomization rate fluctuation range of the same type of atomization piece assembled (such as 01, 02, 03, 04 of the atomization piece 1) is within 10%, which is relatively stable.

Test 2: Vibration node test based on microporous membrane

### 1. Instrument, Tested Sample:

Instrument: Doppler laser vibrometer (specification model: LV-FS01, manufacturer: Sunny Optical); signal generator (specification: AFG10022, manufacturer: Tektronix), power amplifier (specification: ATA-2031, manufacturer: Antai);
Tested sample: The diameter of the microporous area of the microporous membrane of the tested sample was 4 mm, and the outer ring area was an annular area with an outer diameter of 8 mm and an inner diameter of 4 mm;

### 2. Experimental Steps

Atomization piece 3 was connected with the signal source and power amplifier, the photoelectric spot emitted by the optical lens of the Doppler laser vibrometer was moved to the center of the microhole piece, then the height of the lens was adjusted so that the received light intensity reached more than 80%. The output mode of the signal source was adjusted to frequency sweep mode output, the output voltage RMS was 6V, and the frequency sweep range was 1-200 KHz. Then the vibration was measured and the resonant frequency of the atomization piece 3 in the target frequency band was calculated; this resonant frequency was used as the fixed frequency output frequency of the signal source, the signal was output and the output voltage RMS was 6V. The test point was to take the center of the microporous membrane 32 as the coordinate axis 0 point, and the vibration was tested at every 0.25 mm along the positive and negative directions of the X axis and Y, the displacement of one point and two adjacent points was used to make a circle to calculate the surface curvature;

3. The experimental results are shown in Table 2.

TABLE 2

| X-axis | Displace- | | Y-axis | Displace- | |
|---|---|---|---|---|---|
| coordinate | ment | Surface | coordinate | ment | Surface |
| mm | μm | curvature | mm | μm | curvature |
| −3.00 | 0.71 | — | 3.00 | 0.42 | — |
| −2.75 | 0.44 | 0.43 | 2.75 | 0.18 | 1.85 |
| −2.50 | 0.24 | 3.70 | 2.50 | 0.13 | 3.13 |
| −2.25 | 0.64 | 0.20 | 2.25 | 0.46 | 1.35 |
| −2.00 | 0.97 | 0.25 | 2.00 | 0.58 | 3.13 |
| −1.75 | 1.24 | 2.08 | 1.75 | 0.46 | 1.49 |
| −1.50 | 1.28 | 1.39 | 1.50 | 0.05 | 3.70 |
| −1.25 | 1.23 | 0.43 | 1.25 | 0.31 | 0.10 |
| −1.00 | 1.15 | 1.49 | 1.00 | 0.59 | 0.34 |
| −0.75 | 0.91 | 2.13 | 0.75 | 0.81 | 0.68 |
| −0.50 | 0.88 | 0.79 | 0.50 | 0.95 | 1.30 |
| −0.25 | 0.9 | 0.89 | 0.25 | 0.99 | 0.16 |
| 0.00 | 0.98 | 0.29 | 0.00 | 1.02 | 0.32 |
| 0.25 | 1.04 | 2.70 | −0.25 | 1.03 | 1.49 |
| 0.50 | 0.9 | 0.29 | −0.50 | 0.94 | 1.30 |
| 0.75 | 0.73 | 1.85 | −0.75 | 0.71 | 0.27 |
| 1.00 | 0.71 | 2.78 | −1.00 | 0.43 | 2.33 |
| 1.25 | 1.16 | 0.08 | −1.25 | 1.25 | 2.33 |
| 1.50 | 1.57 | 1.23 | −1.50 | 0.67 | 3.03 |
| 1.75 | 1.72 | 3.57 | −1.75 | 0.84 | 2.86 |
| 2.00 | 1.55 | 1.16 | −2.00 | 0.78 | 2.04 |
| 2.25 | 0.83 | 0.14 | −2.25 | 0.27 | 1.47 |
| 2.50 | 0.28 | 3.23 | −2.50 | 0.14 | 3.33 |
| 2.75 | 0.58 | 0.19 | −2.75 | 0.6 | 1.14 |
| 3.00 | 0.84 | — | −3.00 | 0.77 | — |

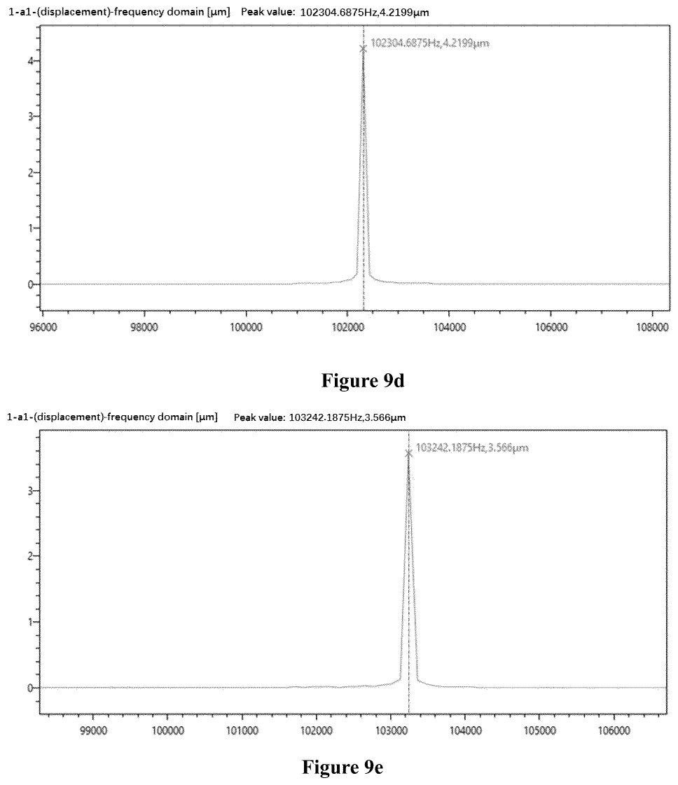

Specifically, the diagram drawn based on the above results is shown in FIG. 8. According to FIG. 8, it can be seen that the nodes of the sample are all within ±0.25 mm of the boundary between area 1 and area 2.

### Test 3: Vibration Stability Test of the Atomization Module

### 1. Instrument, Tested Sample:

Instrument: Doppler laser vibrometer (specification model: LV-FS01, manufacturer: Sunny Optical); signal generator (specification: AFG10022, manufacturer: Tektronix), power amplifier (specification: ATA-2031, manufacturer: Antai);
Tested sample: The sample was assembled in the above manner, the structure of the atomizer of the test sample was shown in FIG. 1, the atomization piece sample was the structure of FIG. 2, and the center position of the microporous membrane 32 was tested;

### 2. Experimental Steps

Atomization piece 3 was connected with the signal source and power amplifier, the photoelectric spot emitted by the optical lens of the Doppler laser vibrometer was moved to the center of the microhole piece, then the height of the lens was adjusted so that the received intensity reached more than 80%. The output mode of the signal source was adjusted to frequency sweep mode output, the output voltage RMS was 6V, and the frequency sweep range was 1-200 KHz. Then the vibration was measured and the resonant frequency of the atomization piece 3 in the target frequency band was calculated; this resonant frequency was used as the fixed frequency output frequency of the signal source, the output voltage RMS was 6V and the signal was output. The test point was the center of the microporous membrane 32, and the test was continued at a fixed frequency for more than 3 S;

### 3. Experimental Results

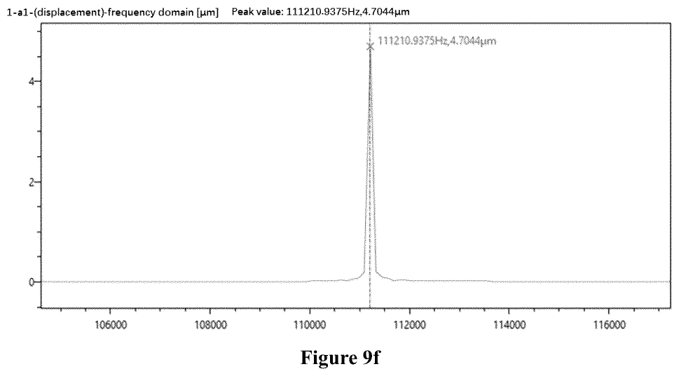

The results of the center displacement of micropore area 321 in atomization modules 1, 2, and 3-time domain (μm), and the spectrum diagrams after Fourier transform are shown in FIG. 9, wherein FIGS. 9a, 9b, and 9c are the results of the center displacement of micropore area 321 in atomization modules 1, 2, and 3-time domain (μm), FIGS. 9d, 9e, and 9f are the spectrum diagrams after Fourier transform of micropore area 321 in atomization modules 1, 2, and 3 respectively. As shown in FIG. 9, the vibration of the center of the microporous piece was monitored for 3-5 s, and the vibration of the three samples was stable.

### Test 4: Spray Amount Test and Delivery Dose Uniformity Test of Tiotropium Bromide

Preparation of tiotropium bromide preparations: the excipients and raw materials were weighed into a 500 ml beaker according to the prescription table, 95% water for injection of the solution weight was added, the mixture was stirred magnetically to completely dissolve the raw materials and excipients; the pH was adjusted to 2.8 with 3.7% hydrochloric acid solution; then water for injection was added to full volume. The medicinal solution in Table 3 was dispensed into the vials matched with the atomizer and stored for later use.

TABLE 3
Prescription table

| Prescription ingredients | Function | Amount |
|---|---|---|
| Tiotropium bromide | Main drug | 27.25 mg |
| Disodium edetate | Metal ion chelating agent | 10 mg |
| Benzalkonium chloride | Preservative | 10 mg |
| Hydrochloric acid | pH regulator | Appropriate amount |
| Water for Injection | Solvent | 100 ml |

Spray amount test of different commercially available atomizers using tiotropium bromide:

1. Instruments: electronic balance (specification: XPR404S, manufacturer: Mettler, Switzerland), signal generator (specification: AFG10022, manufacturer: Tektronix), power amplifier (specification: ATA-2031, manufacturer: Antai);

### 2. Experimental Steps:

Testing sample:
Sample 1: The atomization module was assembled according to the atomizer drawing parts of the above embodiment;
Sample 2: Aerogen solo
Sample 3: Pair eflow

The samples were connected with the signal source and power amplifier and the output parameters were adjusted;

The signal source was used to drive and find the maximum driving frequency, then the maximum driving frequency was used and the output voltage RMS was 25V, and the atomization device was continuously atomized for 2 min;

After standing for 10 min, the maximum driving frequency was used, the output voltage RMS was 25V, and 10 spray volumes in parallel were tested;

The spray amount for 5 consecutive days was tested, and 10 sprays every day;

3. The experimental results are shown in Table 4.

TABLE 4

| | Driving | Days/Sprayed | | | | | | | | | | | AVE on |
|---|---|---|---|---|---|---|---|---|---|---|---|---|---|
| | frequency | amount | | | | | | | | | | | the day |
| Device | KHz | mg | 1 | 2 | 3 | 4 | 5 | 6 | 7 | 8 | 9 | 10 | (mg) |
| Atomization | 101 | 1 | 21.3 | 21.8 | 21.8 | 22.0 | 22.2 | 22.2 | 22.4 | 21.9 | 22.0 | 22.2 | 22.0 |
| module-01 | | 2 | 21.7 | 22.0 | 22.5 | 22.9 | 22.4 | 21.3 | 22.2 | 22.2 | 21.2 | 22.3 | 22.1 |
| | | 3 | 20.3 | 21.1 | 19.9 | 20.4 | 20.9 | 20.1 | 20.9 | 20.1 | 20.8 | 20.5 | 20.5 |
| | | 4 | 20.5 | 21.5 | 20.6 | 20.9 | 21.3 | 22.3 | 22.6 | 21.7 | 21.4 | 21.7 | 21.5 |
| | | 5 | 21.2 | 22.0 | 21.3 | 21.9 | 21.7 | 21.6 | 21.3 | 21.2 | 22.0 | 21.5 | 21.6 |
| | | 5-Day single value | 106.5% | | | | | | | | | | |
| | | MAX of medicinal | | | | | | | | | | | |
| | | solution (%) | | | | | | | | | | | |
| | | 5-Day single value | 92.6% | | | | | | | | | | |
| | | MIN of medicinal | | | | | | | | | | | |
| | | solution (%) | | | | | | | | | | | |
| | | 5-Day average | 21.5 | | | | | | | | | | |
| | | AVE of medicinal | | | | | | | | | | | |
| | | solution (mg) | | | | | | | | | | | |
| | | 5-Day average | 102.6% | | | | | | | | | | |
| | | MAX of medicinal | | | | | | | | | | | |
| | | solution (mg) | | | | | | | | | | | |
| | | 5-Day average | 95.3% | | | | | | | | | | |
| | | MIN of medicinal | | | | | | | | | | | |
| | | solution (mg) | | | | | | | | | | | |
| Atomization | 102 | 1 | 21.0 | 21.4 | 21.8 | 21.8 | 22.0 | 22.3 | 22.7 | 22.6 | 22.8 | 22.8 | 22.1 |
| module-02 | | 2 | 20.8 | 21.4 | 22.0 | 22.0 | 22.6 | 22.5 | 22.8 | 23.3 | 22.6 | 22.6 | 22.3 |
| | | 3 | 20.2 | 21.0 | 20.5 | 20.7 | 21.4 | 21.2 | 21.1 | 21.4 | 21.6 | 21.7 | 21.1 |
| | | 4 | 21.8 | 21.9 | 21.9 | 22.0 | 20.9 | 21.2 | 21.5 | 21.2 | 21.5 | 22.0 | 21.6 |
| | | 5 | 21.7 | 21.6 | 21.9 | 21.8 | 22.1 | 22.2 | 22.1 | 22.1 | 22.2 | 21.8 | 22.0 |
| | | 5-Day single value | 107.1% | | | | | | | | | | |
| | | MAX of medicinal | | | | | | | | | | | |
| | | solution (%) | | | | | | | | | | | |
| | | 5-Day single value | 92.8% | | | | | | | | | | |
| | | MIN of medicinal | | | | | | | | | | | |
| | | solution (%) | | | | | | | | | | | |
| | | 5-Day average | 21.8 | | | | | | | | | | |
| | | AVE of medicinal | | | | | | | | | | | |
| | | solution (mg) | | | | | | | | | | | |
| | | 5-Day average | 102.1% | | | | | | | | | | |
| | | MAX of medicinal | | | | | | | | | | | |
| | | solution (mg) | | | | | | | | | | | |
| | | 5-Day average | 96.7% | | | | | | | | | | |
| | | MIN of medicinal | | | | | | | | | | | |
| | | solution (mg) | | | | | | | | | | | |
| Atomization | 102 | 1 | 20.5 | 20.6 | 20.7 | 21.1 | 21.5 | 21.5 | 21.0 | 21.5 | 21.1 | 21.3 | 21.1 |
| module-03 | | 2 | 19.2 | 19.8 | 20.0 | 19.9 | 20.0 | 20.2 | 20.3 | 20.7 | 20.8 | 20.8 | 20.2 |
| | | 3 | 20.6 | 20.6 | 20.0 | 20.5 | 20.5 | 19.8 | 20.2 | 20.5 | 20.4 | 20.7 | 20.4 |
| | | 4 | 19.6 | 19.5 | 20.6 | 19.9 | 20.1 | 19.6 | 20.1 | 19.9 | 20.3 | 20.2 | 20.0 |
| | | 5 | 21.0 | 20.4 | 20.4 | 21.0 | 20.7 | 21.0 | 20.8 | 21.4 | 20.9 | 20.8 | 20.8 |
| | | 5-Day single value | 104.9% | | | | | | | | | | |
| | | MAX of medicinal | | | | | | | | | | | |
| | | solution (%) | | | | | | | | | | | |
| | | 5-Day single value | 93.7% | | | | | | | | | | |
| | | MIN of medicinal | | | | | | | | | | | |
| | | solution (%) | | | | | | | | | | | |
| | | 5-Day average | 20.5 | | | | | | | | | | |
| | | AVE of medicinal | | | | | | | | | | | |
| | | solution (mg) | | | | | | | | | | | |
| | | 5-Day average | 102.9% | | | | | | | | | | |
| | | MAX of medicinal | | | | | | | | | | | |
| | | solution (mg) | | | | | | | | | | | |
| | | 5-Day average | 97.5% | | | | | | | | | | |
| | | MIN of medicinal | | | | | | | | | | | |
| | | solution (mg) | | | | | | | | | | | |
| Atomization | 102 | 1 | 20.3 | 20.9 | 20.3 | 21.5 | 21.8 | 22.0 | 21.7 | 21.2 | 21.7 | 21.8 | 21.3 |
| module-04 | | 2 | 20.3 | 20.2 | 20.5 | 20.4 | 20.7 | 20.9 | 20.9 | 21.1 | 20.7 | 21.0 | 20.7 |
| | | 3 | 20.8 | 20.3 | 20.7 | 20.4 | 20.9 | 21.2 | 21.1 | 21.1 | 20.9 | 20.8 | 20.8 |
| | | 4 | 20.5 | 21.2 | 20.4 | 20.8 | 20.4 | 21.1 | 21.1 | 21.0 | 21.3 | 21.4 | 20.9 |
| | | 5 | 19.5 | 19.9 | 20.9 | 20.0 | 20.7 | 20.8 | 20.8 | 20.2 | 20.5 | 20.2 | 20.4 |
| | | 5-Day single value | 105.1% | | | | | | | | | | |
| | | MAX of medicinal | | | | | | | | | | | |
| | | solution (%) | | | | | | | | | | | |
| | | 5-Day single value | 96.5% | | | | | | | | | | |
| | | MIN of medicinal | | | | | | | | | | | |
| | | solution (%) | | | | | | | | | | | |
| | | 5-Day average | 20.9 | | | | | | | | | | |
| | | AVE of medicinal | | | | | | | | | | | |
| | | solution (mg) | | | | | | | | | | | |
| | | 5-Day average | 102.4% | | | | | | | | | | |
| | | MAX of medicinal | | | | | | | | | | | |
| | | solution (mg) | | | | | | | | | | | |
| | | 5-Day average | 97.8% | | | | | | | | | | |
| | | MIN of medicinal | | | | | | | | | | | |
| | | solution (mg) | | | | | | | | | | | |
| Parieflow-01 | 117 KHz | 1 | 17.5 | 16.9 | 18.6 | 17.1 | 17.6 | 18.0 | 17.4 | 18.0 | 19.0 | 18.5 | 17.9 |
| | | 2 | 16.8 | 17.1 | 17.9 | 17.7 | 17.7 | 17.8 | 18.2 | 18.6 | 18.3 | 19.3 | 17.9 |
| | | 3 | 20.6 | 20.5 | 20.9 | 20.9 | 21.1 | 19.3 | 19.7 | 21.1 | 22.2 | 21.4 | 20.8 |
| | | 4 | 18.1 | 18.3 | 18.7 | 18.6 | 18.7 | 18.9 | 18.6 | 19.1 | 19.1 | 18.7 | 18.7 |
| | | 5 | 16.8 | 17.1 | 17.0 | 17.5 | 17.6 | 18.0 | 17.9 | 18.0 | 18.2 | 18.7 | 17.7 |
| | | 5-Day single value | 117.9% | | | | | | | | | | |
| | | MAX of medicinal | | | | | | | | | | | |
| | | solution (%) | | | | | | | | | | | |
| | | 5-Day single value | 89.1% | | | | | | | | | | |
| | | MIN of medicinal | | | | | | | | | | | |
| | | solution (%) | | | | | | | | | | | |
| | | 5-Day average | 18.8 | | | | | | | | | | |
| | | AVE of medicinal | | | | | | | | | | | |
| | | solution (mg) | | | | | | | | | | | |
| | | 5-Day average | 111.8% | | | | | | | | | | |
| | | MAX of medicinal | | | | | | | | | | | |
| | | solution (mg) | | | | | | | | | | | |
| | | 5-Day average | 95.1% | | | | | | | | | | |
| | | MIN of medicinal | | | | | | | | | | | |
| | | solution (mg) | | | | | | | | | | | |
| Parieflow-02 | 117 KHz | 1 | 12.5 | 13.8 | 14.4 | 14.6 | 14.3 | 15.2 | 15.9 | 15.4 | 15.0 | 15.1 | 14.6 |
| | | 2 | 11.7 | 10.9 | 11.1 | 11.4 | 11.5 | 12.4 | 11.6 | 11.7 | 12.0 | 12.9 | 11.7 |
| | | 3 | 11.1 | 12.4 | 12.8 | 12.7 | 12.9 | 12.0 | 12.7 | 12.8 | 12.8 | 12.8 | 12.5 |
| | | 4 | 12.9 | 11.6 | 11.9 | 12.9 | 11.5 | 11.7 | 11.8 | 12.6 | 11.7 | 11.6 | 12.0 |
| | | 5 | 11.5 | 11.3 | 11.7 | 12.2 | 11.0 | 11.5 | 11.4 | 13.4 | 13.6 | 12.3 | 12.0 |
| | | 5-Day single value | 124.9% | | | | | | | | | | |
| | | MAX of medicinal | | | | | | | | | | | |
| | | solution (%) | | | | | | | | | | | |
| | | 5-Day single value | 86.0% | | | | | | | | | | |
| | | MIN of medicinal | | | | | | | | | | | |
| | | solution (%) | | | | | | | | | | | |
| | | 5-Day average | 12.7 | | | | | | | | | | |
| | | AVE of medicinal | | | | | | | | | | | |
| | | solution (mg) | | | | | | | | | | | |
| | | 5-Day average | 116.3% | | | | | | | | | | |
| | | MAX of medicinal | | | | | | | | | | | |
| | | solution (mg) | | | | | | | | | | | |
| | | 5-Day average | 93.3% | | | | | | | | | | |
| | | MIN of medicinal | | | | | | | | | | | |
| | | solution (mg) | | | | | | | | | | | |
| Aerogensolo-01 | 128 KHz | 1 | 10.3 | 10.6 | 10.3 | 10.8 | 10.9 | 11.1 | 11.0 | 11.1 | 11.2 | 11.3 | 10.9 |
| | | 2 | 9.9 | 10.7 | 11.1 | 11.2 | 11.4 | 11.6 | 11.4 | 11.6 | 11.4 | 11.5 | 11.2 |
| | | 3 | 10.5 | 11.3 | 11.3 | 11.4 | 11.4 | 11.6 | 11.5 | 11.7 | 11.7 | 11.8 | 11.4 |
| | | 4 | 11.4 | 11.9 | 12.1 | 12.1 | 12.0 | 12.2 | 12.3 | 12.3 | 12.5 | 12.5 | 12.1 |
| | | 5 | 12.4 | 12.3 | 12.0 | 11.6 | 12.9 | 13.0 | 12.6 | 13.1 | 13.1 | 13.3 | 12.6 |
| | | 5-Day single value | 110.1% | | | | | | | | | | |
| | | MAX of medicinal | | | | | | | | | | | |
| | | solution (%) | | | | | | | | | | | |
| | | 5-Day single value | 86.7% | | | | | | | | | | |
| | | MIN of medicinal | | | | | | | | | | | |
| | | solution (%) | | | | | | | | | | | |
| | | 5-Day average | 11.4 | | | | | | | | | | |
| | | AVE of medicinal | | | | | | | | | | | |
| | | solution (mg) | | | | | | | | | | | |
| | | 5-Day average | 108.4% | | | | | | | | | | |
| | | MAX of medicinal | | | | | | | | | | | |
| | | solution (mg) | | | | | | | | | | | |
| | | 5-Day average | 93.3% | | | | | | | | | | |
| | | MIN of medicinal | | | | | | | | | | | |
| | | solution (mg) | | | | | | | | | | | |
| Aerogensolo-02 | 128 KHz | 1 | 10.5 | 11.3 | 11.5 | 11.6 | 11.7 | 11.7 | 12.1 | 11.7 | 12.2 | 12.5 | 11.7 |
| | | 2 | 11.6 | 11.8 | 12.1 | 12.2 | 12.2 | 12.5 | 12.6 | 12.6 | 12.9 | 12.9 | 12.3 |
| | | 3 | 13.2 | 13.5 | 13.7 | 13.7 | 14.0 | 14.0 | 14.0 | 14.1 | 14.0 | 14.3 | 13.8 |
| | | 4 | 10.8 | 12.3 | 12.4 | 12.4 | 12.6 | 12.7 | 12.7 | 12.7 | 12.8 | 12.8 | 12.4 |
| | | 5 | 12.2 | 10.9 | 12.8 | 12.8 | 12.9 | 13.0 | 13.0 | 13.2 | 13.2 | 13.2 | 12.7 |
| | | 5-Day single value | 113.5% | | | | | | | | | | |
| | | MAX of medicinal | | | | | | | | | | | |
| | | solution (%) | | | | | | | | | | | |
| | | 5-Day single value | 83.3% | | | | | | | | | | |
| | | MIN of medicinal | | | | | | | | | | | |
| | | solution (%) | | | | | | | | | | | |
| | | 5-Day average | 12.6 | | | | | | | | | | |
| | | AVE of medicinal | | | | | | | | | | | |
| | | solution (mg) | | | | | | | | | | | |
| | | 5-Day average | 109.9% | | | | | | | | | | |
| | | MAX of medicinal | | | | | | | | | | | |
| | | solution (mg) | | | | | | | | | | | |
| | | 5-Day average | 92.8% | | | | | | | | | | |
| | | MIN of medicinal | | | | | | | | | | | |
| | | solution (mg) | | | | | | | | | | | |
| Aerogensolo-03 | 128 KHz | 1 | 9.0 | 9.7 | 9.6 | 9.4 | 9.7 | 9.8 | 9.6 | 9.4 | 9.7 | 9.8 | 9.6 |
| | | 2 | 10.1 | 9.7 | 10.0 | 9.4 | 9.4 | 9.5 | 9.7 | 9.7 | 9.7 | 9.3 | 9.7 |
| | | 3 | 8.4 | 9.0 | 8.6 | 9.9 | 9.6 | 8.6 | 9.3 | 9.5 | 9.1 | 9.2 | 9.1 |
| | | 4 | 8.2 | 9.1 | 9.6 | 9.2 | 9.3 | 9.5 | 9.5 | 8.8 | 9.5 | 9.3 | 9.2 |
| | | 5 | 7.3 | 9.7 | 9.3 | 9.9 | 9.8 | 9.4 | 9.4 | 9.5 | 9.6 | 9.4 | 9.3 |

| 5-Day single value | 107.8% |
|---|---|
| MAX of medicinal | |
| solution (%) | |
| 5-Day single value | 87.4% |
| MIN of medicinal | |
| solution (%) | |
| 5-Day average | 9.4 |
| AVE of medicinal | |
| solution (mg) | |
| 5-Day average | 103.0% |
| MAX of medicinal | |
| solution (mg) | |
| 5-Day average | 97.4% |
| MIN of medicinal | |
| solution (mg) | |

Conclusion: The 5-day average spray amount of sample 1 is within the range of the average ±10%, and the single value of the daily spray amount is within the range of the 5-day average ±10%. The sample spray amount is stable and achieves the effect of quantitative drug administration. Compared with sample 1, sample 2 and sample 3 have single values or average values outside the range of +10%, and the spray amount stabilities are worse than sample 1.

### Test 5: Delivered Dose Uniformity Test of Tiotropium Bromide:

### 1. Instruments, Reagents and Reference Samples

Instruments: electronic balance (manufacturer: Mettler, Switzerland), signal generator (specification: AFG10022, manufacturer: Tektronix), power amplifier (specification: ATA-2031, manufacturer: Antai), liquid chromatography: Agilent HPLC-1260, chromatograph Column: NanochromChromCore 120C8 150*4.6 mm 5 μm, drug particle collector—Copley scientific LCP 5 (manufacturer: Copley);

Reagents: the diluent was a solution of disodium ethylenediaminetetra acetate in hydrochloric acid, the mobile phase was 0.18% sodium heptane sulfonate solution, acetonitrile;

### 2. Experimental Steps

A suitable tip adapter was used to ensure that the spray tip port was flush with the sample collection tube port. A circular filter paper with 25 mm diameter was put into the base and fixed on one end of the sample collection tube. The base port was connected with the vacuum pump, and the other end of the sample collection tube was connected with the flow meter, the vacuum pump was adjusted so that the air was extracted from the sample collection tube (including filter paper) at a flow rate of 28.3 L/min (+5%). When the measuring device and the spray to be measured were connected for measurement, air should be continuously extracted from the device to avoid loss of active substances into the air. The connections between the components of the assembled device should be airtight, with all air extracted from the sample collection tube passing only through the inhalation spray to be tested.

Delivery dose uniformity test in the tub: The power of the signal generator and high-voltage amplifier was turned on, and the measurement frequency and amplitude required for the atomization piece 3 were set on the signal generator. The output voltage RMS was 25V and the output frequency was 101 KHz. 1 Bottle of the test product was taken and placed on the balance, and the balance was reset to zero, the product was insert into the special tip adapter. After connecting the timer (1.5 s), the vacuum pump was turned on, and the product was sprayed once (the flow rate for air extraction was 5 s), and then sprayed again. The inlet end of the unit dose sampling device was tilted upward 45°, the product (the amount of formulation released per spray was obtained by weighing) was taken out and sealed with a cap, the other end of the unit dose sampling device was tilted upward 45°, then the filter paper was put into the sample collection tube, and an appropriate amount of diluent was immediately and accurately transferred to the unit dose sampling device, the device was covered with a lid and manually rotated left and right to shake, so that the diluent was fully contacted the inner wall of the sample collection tube, and then the device was shaken again, and an appropriate amount was taken using a syringe without a rubber stopper, the mixture was filtered through a membrane, the initial filtrate was discarded, and the subsequent filtrate was taken as the test solution. Ten delivered doses were measured in parallel.

Remarks: 1. Inside the tip for 10 doses of sample 10 and the atomization piece 3 were transferred by 10 ml diluent, respectively. 2. The adapter was transferred to the volumetric flask with an appropriate amount of diluent;

3. The experimental results are shown in Table 5.

TABLE 5

| | | | | | Percentage of |
|---|---|---|---|---|---|
| | | | | | the single |
| | | | Mean | | value of |
| | | Single | value of | | delivered |
| | | value of | delivered | | dose to the |
| Device | | delivered | dose (μg) | Corresponding | mean value |
| number | No. | dose (μg) | AVE ± SD | flow rate | (%) |
| Device 1 | Dose 1 | 8.19 | 8.04 ± 0.24 | 28.3 L/min | 102 |
| | Dose 2 | 8.32 | | | 103 |
| | Dose 3 | 8.08 | | | 100 |
| | Dose 4 | 7.84 | | | 98 |
| | Dose S | 8.08 | | | 100 |
| | Dose 6 | 7.79 | | | 97 |
| | Dose 7 | 8.42 | | | 105 |
| | Dose 8 | 7.76 | | | 97 |
| | Dose 9 | 7.76 | | | 97 |
| | Dose 10 | 8.12 | | | 101 |
| Device 2 | Dose 1 | 7.63 | 7.75 ± 0.25 | 28.3 L/min | 98 |
| | Dose 2 | 8.12 | | | 105 |
| | Dose 3 | 7.63 | | | 98 |
| | Dose 4 | 7.45 | | | 96 |
| | Dose 5 | 7.48 | | | 97 |
| | Dose 6 | 8.10 | | | 105 |
| | Dose 7 | 7.62 | | | 98 |
| | Dose 8 | 8.02 | | | 103 |
| | Dose 9 | 7.61 | | | 98 |
| | Dose 10 | 7.81 | | | 101 |
| Device 3 | Dose 1 | 8.37 | 8.71 ± 0.25 | 28.3 L/min | 96 |
| | Dose 2 | 8.69 | | | 100 |
| | Dose 3 | 8.71 | | | 100 |
| | Dose 4 | 9.18 | | | 105 |
| | Dose 5 | 8.60 | | | 99 |
| | Dose 6 | 8.66 | | | 99 |
| | Dose 7 | 8.68 | | | 100 |
| | Dose 8 | 9.13 | | | 105 |
| | Dose 9 | 8.56 | | | 98 |
| | Dose 10 | 8.54 | | | 98 |
| Device 4 | Dose 1 | 8.39 | 8.66 ± 0.21 | 28.3 L/min | 97 |
| | Dose 2 | 8.43 | | | 97 |
| | Dose 3 | 8.49 | | | 98 |
| | Dose 4 | 8.49 | | | 98 |
| | Dose 5 | 8.59 | | | 99 |
| | Dose 6 | 8.81 | | | 102 |
| | Dose 7 | 8.73 | | | 101 |
| | Dose 8 | 8.78 | | | 101 |
| | Dose 9 | 8.92 | | | 103 |
| | Dose 10 | 8.96 | | | 103 |

Conclusion: The single values of sample continuously delivered 10 doses are all within the average ±5% range, the delivered dose is stable and achieves quantitative dosing effect.

Test 6: Particle size test of samples with different atomization amounts

Instruments: signal generator (specification: AFG10022, manufacturer: Tektronix), power amplifier (specification: ATA-2031, manufacturer: Antai), Malvern laser particle size analyzer

### Experimental Steps:

Tested sample: According to the atomizer drawing parts of the above embodiment, the atomization module was assembled

Test: The absorption pool channel and laser particle sizer were assembled, a 25 mm diameter circular filter paper was placed in the base, and fixed on one end of the sample collection tube. The base port was connected with the vacuum pump, the other end of the sample collection tube was connected with the air outlet under the absorption pool, the flow meter was connected with the air inlet on the artificial throat. Then the pump and flow meter were turned on, and the vacuum pump was adjusted so that the air was extracted from the sample collection tube (including filter paper) at a flow rate of 28.3 L/min (±5%). After the flow rate measurement was completed, the flow meter was removed. The sample was connected with the air inlet of the artificial throat through the adapter when the pump is running, the power of the signal generator and high-voltage amplifier was turned on, and the measurement frequency and amplitude required for the atomization piece 3 were set on the signal generator. The amplification factor 42 was set on the amplifier, the final output voltage RMS was 25V, the output frequency was 101 KHz, and then the spray test was conducted.

### Results

| Atomization | ≤5.8 μm | ≤5.8 μm | | | |
|---|---|---|---|---|---|
| amount μL/s | % min | % max | Range | SD | average |
| 10.7 | 76.37 | 79.01 | 2.63 | 1.15 | 77.61 |
| 12 | 73.12 | 80.48 | 7.36 | 1.93 | 76.67 |
| 15.3 | 67.99 | 86.07 | 18.08 | 2.85 | 75.90 |
| 18 | 64.30 | 75.98 | 11.68 | 2.91 | 69.68 |
| 20 | 64.88 | 67.21 | 2.33 | 1.03 | 66.27 |

Finally, it should be noted that: the above embodiments are only used to illustrate the technical solution of the present invention, but not to limit it. Although the present invention has been described in detail with reference to the foregoing embodiments, those of ordinary skill in the art should understand that: the technical solutions described in the foregoing embodiments can still be modified, or some or all of the technical features can be equivalently replaced; and these modifications or substitutions do not cause the essence of the corresponding technical solution to depart from the scope of the technical solution of each embodiment of the present invention.

1-25. (canceled)

26. A metered-dose atomizer comprising a metered-dose atomization module of the atomizer, wherein the metered-dose atomizer is an atomizer that can quantitatively atomize 5 μL-60 μL in 1 s-3 s.

27. The metered-dose atomizer of claim 26, wherein the percentage of droplets with an aerodynamic particle size of less than 5.8 μm in the atomizer to the total mass of the droplets is more than 65%.

28. The metered-dose atomizer of claim 26, wherein the metered-dose atomization module comprises:
a spray head, providing with an atomization cavity with a step surface;
an atomization piece, which comprises an annular brake and a microporous membrane attached to the side surface of the annular brake;
rubber rings, which comprise a first sealing ring and a second sealing ring, the first sealing ring and the second sealing ring are both annular and clamped on both sides of the atomization piece respectively;
and a pressing plate, which is annular and fixed with the spray head, and is adapted to cooperate with the step surface to install the atomization piece and the rubber rings in the atomization cavity.

29. The metered-dose atomizer of claim 26, wherein the microporous membrane of the metered-dose atomization module comprises a central area covering the central hole of the annular brake, and an adhesive area connected to the annular brake; and the internal stress of the microporous membrane remains constant before and after connecting.

30. The metered-dose atomizer of claim 26, wherein the central area of the metered-dose atomization module comprises a micropore area densely covered with micropores, and an outer ring area surrounding the periphery of the micropore area, the total surface curvature change of the micropore area is larger than that of the outer ring area.

31. The metered-dose atomizer of claim 26, wherein at least one of:
(i) the microporous membrane of the metered-dose atomization module is made of stainless steel material, and the Vickers hardness of the stainless steel material is 200-500 HV;
(ii) the annular brake of the metered-dose atomization module is made of piezoelectric ceramic, the piezoelectric constant of the piezoelectric ceramic is 200-800 pC/N, and the mechanical quality factor is 50-1200; and
(iii) the upper and lower surfaces of the first sealing ring of the metered-dose atomization module are both provided with annular protrusions, and the protrusions are suitable for sealing with the step surface of the atomization cavity or the microporous membrane.

32. The metered-dose atomizer of claim 26, wherein the side surface of the pressing plate of the metered-dose atomization module is further provided with a positioning ring coaxially arranged with the pressing plate; the outer diameter of the positioning ring is larger than or equal to the outer diameter of the second sealing ring, and the inner diameter is smaller than or equal to the inner diameter of the second sealing ring, which is suitable for corresponding press fit with the first sealing ring.

33. The metered-dose atomizer of claim 32, wherein further comprising:
a medicine bottle, which is detachably connected to the inlet of the atomization cavity in the spray head;
a shell, which comprises a curved neck shell and a main shell, the curved neck shell is located on the outside of the medicine bottle, and one end is connected with the spray head, and the other end is connected with the main shell;
and a driving device, which is located in the main shell and is electrically connected with the atomization piece.

34. The metered-dose atomizer of claim 33, wherein the driving frequency of the driving device is the natural frequency of the atomization piece.

35. A spray assembly comprising spray and the metered-dose atomizer of claim 26, wherein the active pharmaceutical ingredient in the spray comprises at least one of β2 receptor agonist, glucocorticoid, muscarinic receptor antagonist and phosphodiesterase 4 inhibitor.

36. The spray assembly of claim 35, wherein at least one of:
(1) the β2 receptor agonist comprises at least one of salbutamol or a pharmaceutically acceptable salt thereof, fenoterol or a pharmaceutically acceptable salt thereof, terbutaline or a pharmaceutically acceptable salt thereof, formoterol or a pharmaceutically acceptable salt thereof, olodaterol or a pharmaceutically acceptable salt thereof, arformoterol or a pharmaceutically acceptable salt thereof, indacaterol or a pharmaceutically acceptable salt thereof, vilanterol or a pharmaceutically acceptable salt thereof;
(2) the glucocorticoids comprise at one least of fluticasone or pharmacologically acceptable salts or esters thereof, mometasone or pharmacologically acceptable salts or esters thereof, ciclesonide or pharmacologically acceptable salts or esters thereof, beclomethasone or pharmacologically acceptable salts or esters thereof, flunisolide or pharmacologically acceptable salts or esters thereof, budesonide or pharmacologically acceptable salts or esters thereof, triamcinolone or pharmacologically acceptable salts or esters thereof, dexamethasone or pharmacologically acceptable salts or esters thereof;
(3) the muscarinic receptor antagonist comprises at one least of tiotropium or pharmaceutically acceptable salts thereof, glycopyrronium or pharmaceutically acceptable salts thereof, umeclidinium or pharmaceutically acceptable salts thereof, aclidinium or pharmaceutically acceptable salts thereof, ipratropium or pharmaceutically acceptable salts thereof, oxitropium or pharmaceutically acceptable salts thereof, revefenacin or pharmaceutically acceptable salts thereof; and
(4) the phosphodiesterase 4 inhibitor comprises at least one of roflumilast or a pharmaceutically acceptable derivative thereof, apremilast or a pharmaceutically acceptable derivative thereof.

37. A method for treating COPD (chronic obstructive pulmonary disease) and/or asthma in a human in need thereof, wherein the method comprises administering to the human a spray assembly of claim 35.

38. A spray assembly comprising spray and a metered-dose atomizer of claim 26, wherein the active pharmaceutical ingredient in the spray comprises at least one of prostacyclin, treprostinil and iloprost.

39. A method for treating pulmonary arterial hypertension in a human in need thereof, wherein the method comprises administering to the human a spray assembly of claim 38.

40. A spray assembly comprising spray and a metered-dose atomizer of claim 26, wherein the active pharmaceutical ingredient in the spray comprises antibiotic or an antiviral drug, the antibiotic comprises at least one of aztreonam, tobramycin, amikacin, and ciprofloxacin, and the antiviral drug comprises at least one of zanamivir, Laninamivir, and ribavirin.

41. A method for treating lung infection in a human in need thereof, wherein the method comprises administering to the human a spray assembly of claim 40.

42. A spray assembly comprising spray and a metered-dose atomizer of claim 26, wherein the active pharmaceutical ingredient in the spray comprises at least one of pirfenidone and nintedanib.

43. A method for treating idiopathic pulmonary fibrosis in a human in need thereof, wherein the method comprises administering to the human a spray assembly of claim 42.

44. A spray assembly comprising spray and a metered-dose atomizer of claim 26, wherein the active pharmaceutical ingredient in the spray comprises a small molecule cytotoxic drug or a biological agent.

45. A method for treating lung cancer in a human in need thereof, wherein the method comprises administering to the human a spray assembly of claim 44.
